(19) United States
(12) Patent Application Publication
(10) Pub. No.: US 2010/0179490 A1
(43) Pub. Date: Jul. 15, 2010

(54) Ear canal rinsing device

(75) Inventors: Kevin Connelly, Chaska, MN (US); Curt Miller, Inver Grove Heights, MN (US); Eric Fraser, Woodbury, MN (US)

Correspondence Address: WESTMAN CHAMPLIN & KELLY, P.A., SUITE 1400, 900 SECOND AVENUE SOUTH, MINNEAPOLIS, MN, 55402, US

(73) Assignee: Micromedics, Inc., St. Paul, MN (US)

(21) Appl. No.: 12/537,729

(22) Filed: Aug. 7, 2009

Related U.S. Application Data
(60) Provisional application No. 61/086,903, filed on Aug. 7, 2008.

(51) Int. Cl. A61M 31/00

(52) U.S. Cl. 604/257

(57) ABSTRACT

An ear canal flushing device includes a main body with a cavity for storing a liquid and a temperature sensor for indicating when the liquid is within a selected temperature range that will not adversely affect the patient when the liquid contacts the tympanic membrane. A nozzle is removably attachable to the main body where the nozzle includes a distal portion adapted for insertion into the ear canal and an internal nozzle lumen in fluid communication with the opening in the main body. The nozzle also includes at least one discharge port in fluid communication with the internal nozzle lumen wherein when pressure is applied to the main body a volume of the cavity decreases and liquid is forced out of the at least one discharge port such that the ear canal can be rinsed.

### CROSS REFERENCE TO RELATED APPLICATION

The present application claims the benefit of U.S. Provisional Patent Application Ser. No. 61/086,903 which was filed on Aug. 7, 2008, the contents of which are incorporated by reference in its entirety.

### FIELD OF THE INVENTION

The present invention generally relates to a hand held device utilized to irrigate and flush a canal or passage in a human body. More particularly, the present invention relates to a device to irrigate and flush debris such as cerumen, or ear wax, from the ear canal.

### BACKGROUND OF THE INVENTION

Many people are afflicted with ear problems that require medical treatment. A typical ear affliction includes the blockage or obstruction of the ear canal which can cause both pain to the tympanic membrane, or ear drum, as well as the loss of hearing.

A typical blockage or obstruction of the ear canal is caused by cerumen, or ear wax. However, the ear canal can also be obstructed by other debris or foreign objects.

The tympanic membrane is sensitive to sound and temperature. The tympanic membrane is also a fragile piece of anatomy that can be easily ruptured if care is not taken while examining and treating the affliction.

Numerous devices have been utilized to remove debris from the ear canal to provide access to the tympanic membrane. One device is an irrigation device which forces a liquid into the ear canal. However, utilizing a typical irrigation device includes the potential of damaging the tympanic membrane because the typical irrigation device directs the fluid inwardly, and in the general direction of the tympanic membrane. If a sufficient amount of pressure is applied to the tympanic membrane by the irrigation device, the tympanic membrane will rupture.

Further, great care must be taken to irrigate the ear canal with a liquid at about body temperature. A liquid below 85° F. and above 110° F. can cause the patient to become disorientated and lose balance due to the difference in temperature between the two ears when the ear being treated is contacted with the liquid.

A mechanical device, such as an ear curette, can also be utilized to remove the obstruction or blockage. However, the use of a mechanical device, such as an ear curette, includes the possibility of damaging the ear drum or tympanic membrane by contacting the end of the mechanical device with the tympanic membrane. For instance an ear curette could be inserted into the ear canal such that the end of the ear curette accidentally punctures the tympanic membrane.

### SUMMARY OF THE INVENTION

The present invention includes an ear canal rinsing device having a main body with a cavity for storing a liquid and a temperature sensor for indicating when the liquid is within a selected temperature range that will not adversely affect the patient when the liquid contacts the tympanic membrane. A nozzle is removably attachable to the main body where the nozzle includes a distal portion adapted for insertion into the ear canal. The nozzle includes an internal nozzle lumen in fluid communication with the opening in the main body. The nozzle also includes at least one discharge port in fluid communication with the internal nozzle lumen wherein when pressure is applied to the main body a volume of the cavity decreases and liquid is forced out of the at least one discharge port such that the ear canal can be rinsed.

### BRIEF SUMMARY OF THE DRAWINGS

FIG. 1 is a perspective view of an ear canal rinsing device.

FIG. 2 is a sectional view of the ear canal rinsing device.

FIG. 3 is a perspective view of a nozzle of the ear canal rinsing device.

FIG. 4 is a top view of the nozzle of the ear canal rinsing device.

FIG. 5 is a side view of the nozzle of the ear canal rinsing device.

FIG. 6 is a sectional view of the nozzle of the ear canal rinsing device along the line A-A in FIG. 5.

### DETAILED DESCRIPTION

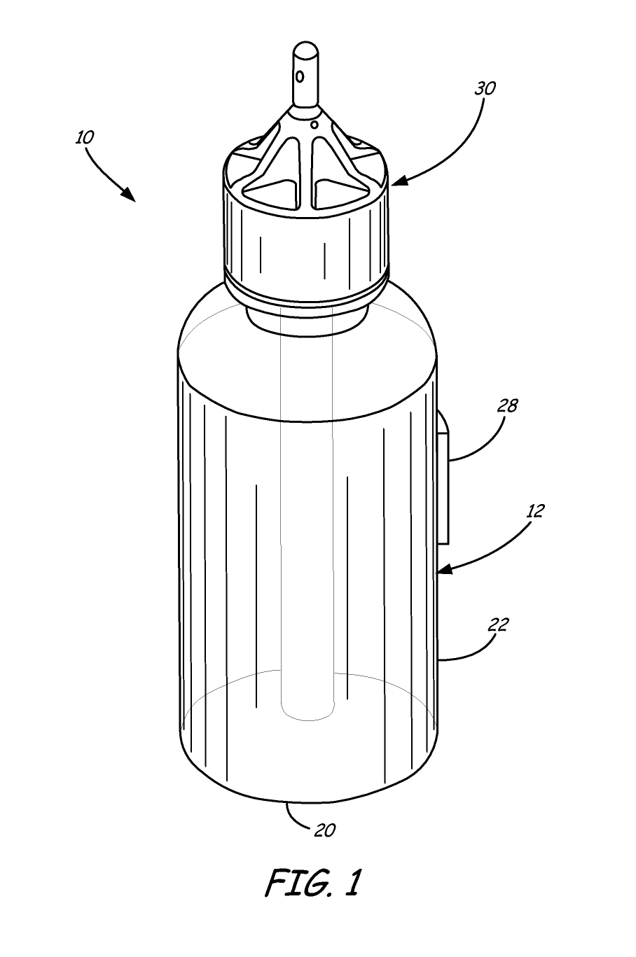

An ear canal rinsing device is generally illustrated in FIG. 1 at 10. The ear canal flushing device includes main body 12 with an opening 14 at a top end 16 to a cavity 18 for storing a liquid. A spray nozzle 30 is removably attached to the top end 16 of the main body where the spray nozzle 30 is inserted into the ear canal and directs the liquid onto the walls of the ear canal.

The device 10 includes a temperature sensor 28 that is attached to the main body 12 that indicates when the temperature of the liquid is within a temperature range that will not adversely affect the patient due to a temperature difference between the tympanic membrane and the liquid. A typical selected temperature range is between about 85° F. and about 110° F.

A typical temperature sensor 28 is a liquid crystal display (LCD) having a pressure sensitive adhesive on one surface where the pressure sensitive adhesive affixes the temperature sensor 28 to an exterior surface of the main body 12. The LCD changes colors when the LCD reaches a temperature within the selected range. While a temperature sensitive LCD is contemplated, other temperature sensor could also be utilized to determine the temperature of the liquid.

The main body 12 includes a bottom wall 20 and a substantially cylindrical side wall 22 extending upwardly from the bottom wall 20. A top wall 23 extends from the side wall 22 where the top wall 23 converges to the opening 14 at the top end 16. The cavity 18 is defined by the bottom wall 20, the side wall 22 and the top wall 23. While a cylindrical configuration of the main body 12 is typical, other configurations of the main body are also contemplated.

The main body 12 also includes a lumen 24 that extends from the top end 16 to proximate the bottom wall 20 where the lumen 24 is located about a parameter of the opening 14. The lumen 24 directs the liquid out of the top end 16 of the main body 12 when opposing portions of the side walls 22 are moved toward each other typically with manual force. As opposing portions of the side wall 22 are moved toward each other, the volume of the cavity 18 is reduced and pressure is applied to the liquid which forces the liquid through the top end 16 of the main body 12.

The main body 12 includes a threaded cylindrical extension 26 which threadably engages a threaded bore 32 in the nozzle 30 to attach the spray nozzle 30 to the main body 12. A bottom portion 38 of the nozzle 30 has a substantially cylindrical outer surface which facilitates gripping of the nozzle 30 when the nozzle 30 is being connected to or disconnected from the main body 12. While a threaded connection is typical, other connecting mechanisms are also contemplated, such as but not limited to a snap connection or a friction engagement.

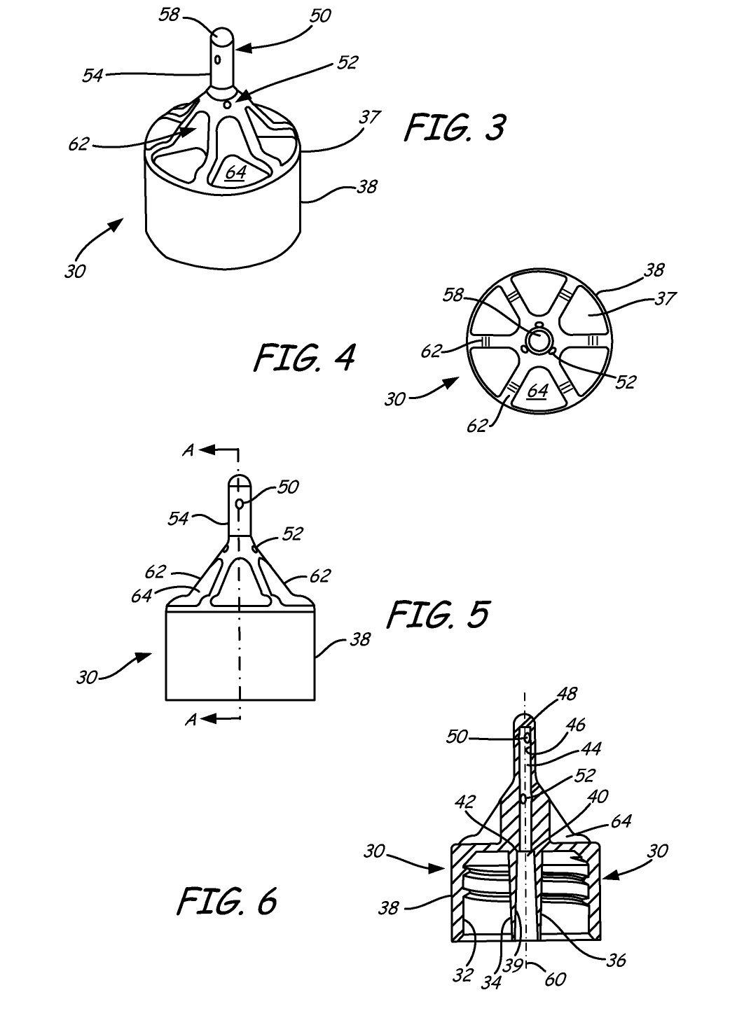

Referring to FIGS. 3-6, the nozzle 30 includes an extension 34 that axially extends from a shoulder and into the threaded bore 32. A distal end of the extension 34 is positioned into the top end of the lumen 24. The extension 34 has a tapered outer surface 36 which decreases in diameter from the shoulder to a distal end of the extension. The extension also decreases in thickness from the shoulder to the distal end of the extension 34.

As the nozzle 30 is threadably secured to the main body 12 through the threaded engagement of the threaded bore 32 and the threaded cylindrical extension 26, the extension 34 is forced into the lumen 24 such that when the nozzle 30 is secured to the main body 12, the outer surface 36 of the extension 34 forms a seal with the lumen 24. The seal prevents liquid from leaking out of the device 10 through the threaded engagement of the nozzle 30 with the main body 12. Leakage through the threaded engagement would detrimentally decrease the pressure of the liquid delivered to the ear canal as well as potentially spraying the care giver or the patient with unwanted liquid.

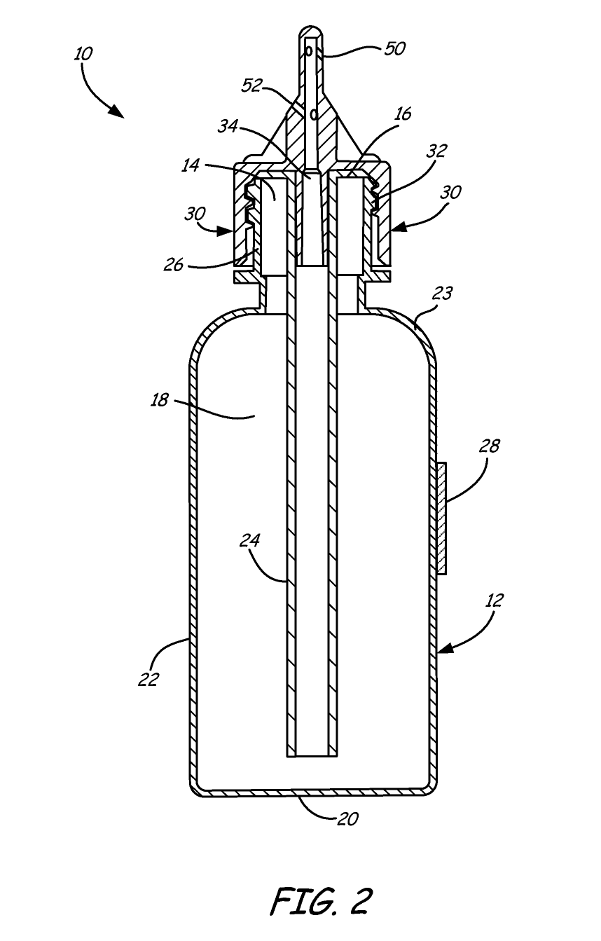

Referring to FIGS. 2 and 6, the extension 34 forms a bottom portion 39 of an interior nozzle lumen 40 that is in fluid communication with the lumen 24. The diameter of the bottom portion 39 gradually decrease to a shoulder 42 that transitions to a top portion 44 of the nozzle lumen 40. The gradually decreasing diameter of the bottom portion 39 increases the pressure on the liquid such that the liquid is delivered to the ear canal at a desired pressure. The top portion 44 of the nozzle lumen 40 has a substantially cylindrical wall 46 and a terminating wall 48 such that the pressure on the liquid is substantially consistent throughout the top portion 44 of the nozzle lumen 40.

Referring to FIGS. 3-6, the fluid exits the top portion 44 of the nozzle lumen 40 through a top row of discharge ports 50 and a bottom row of discharge ports 52. The top row of discharge ports 50 include three evenly spaced ports around the circumference of the top portion 44 of the nozzle lumen that are positioned at about a 45° angle from a central axis 60 of the nozzle 30 such that the liquid being discharged into the ear canal that is closest to the tympanic membrane is forced away from the tympanic membrane and toward the entrance to the ear canal. The angle of the top row of discharge ports can range from about 30° to about 60° or any degree within the range provided that the liquid discharged from the top row of discharge ports 50 does not contact the tympanic membrane.

While a row of three discharge ports is contemplated one or more discharge ports that direct liquid away from the tympanic membrane are also contemplated. Offsetting the top discharge ports such that the top discharge ports do not form a row is also contemplated.

The bottom row of discharge ports 52 includes three evenly spaced ports around the circumference of the top portion 44 that are positioned at about a 45° angle from the central axis 60 of the nozzle 30. The discharge ports 52 are angled to direct liquid to contact the canal wall in a direction toward the tympanic membrane but not contacting the tympanic membrane. The angle of the bottom row of discharge ports can range from about 30° to about 60° or any degree within the range provided that the liquid discharged from the top row of discharge ports 50 does not substantially contact the tympanic membrane. However due to the distance of the bottom row of discharge ports 52 from the tympanic membrane and the pressure of the liquid, the tympanic membrane will not be adversely affected by the ear canal rinse.

While a row of three discharge ports is contemplated, one or more discharge ports that discharge liquid toward the tympanic membrane are also contemplated. Offsetting the bottom discharge ports such that the bottom discharge ports do not form a row is also contemplated.

The bottom row of discharge ports 52 are offset from the top row of discharge ports 50 such that the entire circumference of the ear canal is rinsed instead of only a portion of the ear canal. The angle of the discharge ports 50, 52 is best illustrated in FIG. 2.

The top and bottom discharge ports 50 and 52 have diameters that are typically within the range of 0.024 inches and 0.030 inches. However, ports having diameters smaller than 0.024 inches and larger than 0.030 inches are also contemplated provided a sufficient amount of liquid is discharged at a sufficient pressure to effectively rinse the ear canal of the obstruction.

By directing about half of the liquid to contact the canal wall in a direction toward the tympanic membrane, without actually contacting the tympanic membrane with a substantial amount of liquid, and about half of the liquid away from the tympanic membrane, a more effective rinse is created when compared to a nozzle that only directs a liquid in one direction. While not being bound to any theory, it is believed that the cerumen may be loosed from the canal wall by a liquid flush in one direction and is dislodged from the ear canal wall by a liquid flush in the opposite direction.

While two rows of discharge ports are typical, one or more rows of discharge ports are contemplated. Also while three ports per row of discharge ports are typical, two or more ports per row are contemplated. Also a randomized pattern of discharge ports is also contemplated.

The nozzle 30 includes a top portion 54 that is inserted into the ear canal. The top portion 54 extends from a shoulder 37 that connects the top portion 54 to the bottom portion 38. The top portion 54 is of a length that will not contact the tympanic membrane of a person with a typical distance from the entrance to the ear canal to the tympanic membrane. The top portion 56 has a rounded end 58 that prevents damage to the tympanic membrane in the unlikely event that the rounded end 58 contacts the tympanic membrane.

The nozzle 30 also includes a plurality of chamfers 62 that are connected to an outer perimeter of the shoulder 37 and the top portion 56 below the top end 58. The chamfers 62 engage the entrance to the ear canal which also prevents excessive insertion of the top portion 56 into the ear canal. The chamfers 62 are spaced apart which prevents a seal from being formed between the nozzle 30 and the ear canal. A seal at the entrance to the ear canal is undesirable because when a seal is formed and liquid is forced into the ear canal, pressure will be placed upon the tympanic membrane which could cause pain at a minimum, and potentially rupture the tympanic membrane.

The chamfers 62 also provide openings 64 through which the liquid and debris such as cerumen can be drained from the ear canal. While six evenly spaced chamfers are typical, three or more chamfers are also contemplated.

Although the present invention has been described with reference to preferred embodiments, workers skilled in the art will recognize that changes may be made in form and detail without departing from the spirit and scope of the invention.

1. An ear canal rinsing device comprising:
a main body a cavity for containing the liquid and an opening;
a temperature sensor attached to the main body for sensing the temperature of the liquid in the cavity; and
a nozzle removably attachable to the main body, the nozzle comprising:
a distal portion adapted for insertion into the ear canal;
an internal nozzle lumen in fluid communication with the opening in the main body; and
at least one discharge port in fluid communication with the internal nozzle lumen wherein when pressure is applied to the main body a volume of the cavity decreases and liquid is forced out of the at least one discharge port such that the ear canal can be rinsed.

2. The device of claim 1 and wherein the temperature sensor comprises a liquid crystal display that changes colors based upon a sensed temperature wherein the liquid crystal display maintains a selected color throughout a selected temperature range.

3. The device of claim 2 and wherein the liquid crystal display includes a pressure sensitive adhesive on one surface thereof that attaches the liquid crystal display to the main body.

4. The device of claim 2 and wherein the selected temperature range comprises between 85° F. and about 110° F.

5. The device of claim 1 and wherein the main body comprises a lumen extending into the cavity from a perimeter of the opening.

6. The device of claim 5 and wherein the nozzle further comprises:
a bottom portion that engages the main body; and
the distal portion wherein the bottom portion and the distal portion are connected by a shoulder.

7. The device of claim 6 and wherein bottom portion of the nozzle comprises:
a surface defining a through bore wherein the surface removably engages the main body; and
an extension extending from the shoulder and into the through bore and wherein the extension comprises a portion of the internal nozzle lumen and wherein the extension forms a seal with the lumen of the main body.

8. The device of claim 7 and wherein the extension comprises a tapered thickness wherein the thickness decreases from the shoulder to a distal end of the extension.

9. The device of claim 7 and wherein the internal nozzle lumen further comprises a shoulder that decreases a diameter of the internal nozzle lumen at an end of the extension and wherein the shoulder includes an opening to a top portion of the internal nozzle lumen wherein the top portion of the internal nozzle lumen includes a substantially cylindrical wall that terminates at an end wall.

10. The device of claim 1 and wherein the at least one discharge port comprises:
a first row of discharge ports positioned at substantially even intervals about a circumference of the distal end wherein the first row of discharge ports are positioned proximate the main body and at a selected angle away from the main body and towards a distal end of the nozzle; and
a second row of discharge ports positioned at substantially even intervals about a circumference of the distal end wherein the second row of discharge ports are positioned proximate the distal end of the nozzle and at a selected angle away from the distal end of the nozzle and towards the main body.

11. The device of claim 10 and wherein the first row of discharge ports comprises three discharge ports and the second row of discharge ports comprises three discharge ports and wherein the first row of discharge ports are offset from the second set of discharge ports such that the first row of discharge ports and the second set of discharge ports do not axially align.

12. The device of claim 10 and wherein the selected angle for the first set of discharge ports comprises the range of between about 30° to about 60° from an axis of the nozzle and the selected angle for the second set of discharge ports comprises the range of between about 30° to about 60° from an axis of the nozzle.

13. The device of claim 10 and wherein the selected angle for the first set of discharge ports comprises about 45° from an axis of the nozzle and the selected angle for the second set of discharge ports comprises about 45° from an axis of the nozzle.

14. The device of claim 5 and wherein the nozzle further comprises at least on chamfer attached to the shoulder and the distal portion of the nozzle.

15. An ear canal rinsing device comprising:
a main body a cavity for containing the liquid and an opening at a proximal end; and
a nozzle attached to the main body at a proximal end and having a distal end wherein the nozzle comprises:
a first set of outlets proximate the distal end that are in fluid communication with the opening wherein when pressure is applied to the main body liquid is forced out of the first set of outlets and into contact with the surface of the ear canal in a direction toward the entrance to the ear canal; and
a second set of outlets proximate the proximal end and in fluid communication with the opening nozzle wherein when pressure is applied to the main body liquid is also forced out of the second set of outlets and into contact with the ear canal in a direction toward the tympanic membrane such that the surface of the ear canal is rinsed.

16. The device of claim 15 and wherein the main body comprises a lumen extending a surface defining the opening and into the cavity.

17. The device of claim 16 and wherein the nozzle further comprises an extension that sealingly engages the lumen wherein the extension includes a tapered through bore in fluid communication with the first and second set of outlet ports.

18. The device of claim 15 and wherein a diameter of the first and second set of outlet ports are in a range of between about 0.024 inches and about 0.030 inches.

19. The device of claim 15 and wherein the nozzle comprises at least one external chamfer that limits the distance that the nozzle can be inserted to the ear canal.

20. The device of claim 15 and wherein the first and second sets of outlet ports are positioned at about 45° from an axis of the nozzle.
